(19) United States
(12) Patent Application Publication
(10) Pub. No.: US 2013/0110105 A1
(43) Pub. Date: May 2, 2013

(54) CARBON COATED ELECTRODE FOR ELECTROSURGERY AND ITS METHOD OF MANUFACTURE

(71) Applicant: Alexander B. VANKOV, Menlo Park, CA (US)

(72) Inventor: Alexander B. VANKOV, Menlo Park, CA (US)

(73) Assignee: PEAK Surgical, Inc., Palo Alto, CA (US)

(21) Appl. No.: 13/284,662

(22) Filed: Oct. 28, 2011

(51) Int. Cl. A61B 18/14; B05D 3/00; B05D 3/02; B05D 7/00

(52) U.S. Cl. 606/41; 427/2.11

(57) ABSTRACT

An electrode for use in electrosurgery. The active tip or edge of the electrode carries a non-stick (to tissue) coating of carbon or graphite and a protein material. The coating is at least 10 [symbol] m thick and supports a high voltage discharge of at least 1000 volts. The coating is formed by dipping the electrode into a mixture of carbon or graphite powder and a liquid binder, then drying the coating.

### FIELD OF THE INVENTION

This invention relates to electrosurgery generally and more specifically to an electrode for an electrosurgical instrument.

### BACKGROUND

Electrosurgery is a well known technology utilizing an applied electric current to cut, ablate or coagulate human or animal tissue. See U.S. Pat. No. 7,789,879 issued to Daniel V. Palanker et al., incorporated herein in its entirety by reference. Typical electrosurgical devices apply an electrical potential difference or a voltage difference between a cutting electrode and a portion of the patient's grounded body in a monopolar arrangement or between a cutting electrode and a return electrode in bipolar arrangement, to deliver electrical energy to the operative field where tissue is to be treated. The voltage is applied as a continuous train of high frequency pulses, typically in the RF (radio frequency) range.

The operating conditions of electrosurgical devices vary, see the above-referenced patent, in particular a configuration of the cutting electrode is described there whereby a conductive liquid medium surrounding the electrode is heated by the applied electric current to produce a vapor cavity around the cutting portion of the electrode and to ionize a gas inside a vapor cavity to produce a plasma. The presence of the plasma maintains electrical conductivity between the electrodes. The voltage applied between the electrodes is modulated in pulses having a modulation format selected to minimize the size of the vapor cavity, the rate of formation of vapor cavity and heat diffusion into the material as the material is cut with an edge of the cutting portion of the cutting electrode.

The operating principle thereby is based on formation of a thin layer of a plasma along the cutting portion of the cutting electrode. Typically some sort of conductive medium, such as saline solution or normally present bodily fluids, surround the cutting portion of the electrode such that the liquid medium is heated to produce a vapor cavity around the cutting portion. During heating an amount of the medium is vaporized to produce a gas inside a vapor cavity. Since typically the medium is saline solution or bodily fluids, the gas is composed primarily of water vapor. The layer of gas is ionized in the strong electric field or on the cutting electrode to make up the thin layer of plasma. Because the plasma is electrically conductive, it maintains electrical conductivity.

The energizing electrical energy modulation format in that patent includes pulses having a pulse duration in the range of 10 microseconds to 10 milliseconds. Preferably the pulses are composed of minipulses having a minipulse duration in the range of 0.1 to 10 microseconds and an interval ranging from 0.1 to 10 microseconds between the minipulses. Preferably the minipulse duration is selected in the range substantially between 0.2 and 5 microseconds and the interval between them is shorter than a lifetime of the vapor cavity. The peak power of the minipulses can be varied from minipulse to minipulse. Alternately, the minipulses are made up of micropulses where each micropulse has a duration of 0.1 to 1 microsecond.

Preferably the minipulses have alternating polarity, that is exhibit alternating positive and negative polarities. This modulation format limits the amount of charge transferred to the tissue and avoids various adverse tissue reactions such as muscle contractions and electroporation. Additional devices for preventing charge transfer to the biological tissue can be employed in combination with this modulation format or separately when the method is applied in performing electrosurgery. This pulsing regime is not limiting.

Typical peak voltages applied to the electrode exceed 1300 volts, and the temperature of the cutting portion of the electrode is maintained between 40 and 1,000° C.

Other modalities of electrosurgery do not require plasma generation, but sometimes only heat the tissue, for instance to desiccate or coagulate (prevent bleeding).

That patent also describes particular shapes of the electrode and especially its cutting (active) portion in terms of shape and dimensionality. Such electrosurgical devices provide several surgical techniques, including cutting, bleeding control (coagulation), desiccation and tissue ablation. Typically different types of electrodes and energizing regimes are used for various purposes since the amount of energy applied and the type of tissue being worked on differ depending on the surgical technique being used.

### SUMMARY

It is known to provide the exposed (non-insulated) electrode tip or edge, which is the active portion of the electrode, with a non-stick and electrically resistive coating, to prevent undesirable tissue adhesion by the tip or edge. Known electrode coatings are conventional polymers or flouro-polymers. Further, a pyrolitic carbon deposition method is known, see Morrison, Jr. U.S. Pat. No. 4,074,710 incorporated herein by reference in its entirety, which forms a carbon coating on an electrosurgery electrode by burning carbohydrate-containing materials deposited on the electrode. Morrison, Jr. also discloses sputter or vapor depositing a thin film (10,000 Å thick) of carbon on the electrode. He further discloses adhering a coating of ground up graphite in an epoxy or plastic binder to the electrode.

In accordance with the present invention, the electrode tip coating is formed of carbon or graphite together with a collagen or other similar bio-compatible material, referred to here as a “protein.” For instance, this coating is carbon or graphite with a protein material (albumin or collagen or gelatin) binder. As well known, graphite is an allotrope of the element carbon, which refers to the way the atoms bond together. Where used herein, “graphite” refers conventionally to the mineral graphite. “Carbon” refers to, e.g., amorphous carbon. The carbon or graphite coating is electrically conductive and when of graphite, its internal structure defines micro-bridges between graphite particles.

The thickness of the carbon/graphite layer on the surface of the electrode is in the range of 10 μm to 1 mm. A typical thickness is about 50 to 500 μm . This is needed to support an electrical discharge as described above at, e.g., an applied voltage exceeding 1000 volts. At voltage under about 1000 volts, the carbon/graphite layer is electrically resistive, but not over that voltage level. Note that this thickness serves as a thermal insulator that protects the underlying material of the electrode from overheating. Conventional carbon sputtering as in Morrison, Jr. provides a thickness of only 0.1 to 1.0 μm of carbon, which is inadequate for this purpose by an order of magnitude.

The exposed portion of the present electrode thereby carries a non-stick coating, which is carbon or graphite with a protein material. The electrode may be a ball, tube, screen, suction coagulator, forceps or other type. Typically the present electrode is intended for a single use (is disposable) meaning only a single surgical operation.

### DETAILED DESCRIPTION

The type of electrical energy applied in use to the present electrode by the control unit may be, e.g., as described in the above-referenced patent so as to provide plasma type conditions at the electrode tip for tissue cutting, but this is not limiting. In one embodiment, the electrode has a 3.0 mm wide spatula shaped tip mounted on a variable length (extendable) shaft. The electrode is, e.g., stainless steel. An exemplary such electrode is that of the PEAK PlasmaBlade® 3.05 surgical instrument supplied by PEAK Surgical, Inc., of Palo Alto, Calif. which has a telescoping electrode shaft and a spatula shaped electrode tip which is 3 mm wide, and an integrated aspiration feature. This instrument includes the hand unit which supports the electrode.

The present electrode is suitable for a monopolar type device (such as the PEAK PlasmaBlade instrument) whereby the return current path is via a grounding pad or other return electrode affixed to the patient's body remote from the electrosurgical instrument. The present electrode is also suitable for a bipolar type device where the return electrode is located on or near the main electrode and is an integral part of the electrosurgical apparatus, as also well known in the field.

The present electrode coating process first involves providing a mixture of carbon powder or graphite powder (of any convenient particle size) and a binder. The mixture is 1% to 50% powdered carbon or graphite (by weight or volume), preferably about 30% by volume. Suitable graphite is available in the form of bars of graphite sold to artists for sketching. The binder is a solution of a protein or similar material such as albumin, gelatin, collagen or other biocompatible material dissolved in water or other solvent. The biocompatible aspect is because some flakes of the coating will come off the electrode in use and end up in the wound due to electrode arcing. A suitable material is a fine collagen powder such as Neocell type 1 and 3 powder, dissolved in water. In another example, the binder is a 35% solution by volume of albumin in saline solution. Any protein-like binding agent that binds to graphite or carbon powder may be used.

The electrode structure is typically a metal structure with a glass coating. This is conventionally made. Then it is briefly dipped into the mixture. Alternatively, the mixture is painted or smeared onto the bare electrode. Note that typically the edge of the electrode does not take much of the mixture due to the sharpness of the edge, and in any case the mixture even when dried, erodes very quickly from the edge in use. Thereby this protein coating does not interfere with the action of the blade edge in terms of the plasma formation. The coated electrode is then air dried for, e.g., one minute to one hour at an ambient temperature of 200° C. to 300° C., e.g. for 5 minutes at 300° C., or until all the solvent has evaporated. The goal is to eliminate any bubbles in the liquid. Then the coated electrode is placed in an oven for a few seconds to an hour, at a temperature of 200° C. to 600° C. Preferably this baking step is 5 minutes at 350° C. The drying and baking can be combined into one step. Some of the protein material denatures or breaks down during the drying and baking, but at least some denatured protein does remain in the finished coating. Further, the coating and drying steps can be performed more than once, to form multiple layers of the protein on the electrode. The electrode is then cooled in the air and ready for assembly with the associated components of the electrosurgery apparatus.

The resulting coating when viewed under magnification has a somewhat roughened and black, grainy and irregular appearance that is grainer near the edges of the electrode but having excellent anti-stick properties as regards tissue. It is also fairly durable.

This disclosure is illustrative and not limiting. Further modifications to the embodiments disclosed here will be apparent to those skilled in the art in light of this disclosure, and are intended to fall within the scope of the appended claims.

We claim:

1. A method of coating an electrode adapted for electrosurgery, comprising the acts of:
providing a mixture of carbon powder or graphite powder and a binder of a protein material dissolved in a solvent;
applying the mixture to at least a portion of the electrode; and
drying the applied mixture to form a coating of carbon or graphite and the protein material on the electrode, wherein the dried coating is at least 10 μm thick and supports a high voltage discharge of at least 1000 volts.

2. The method of claim 1, wherein the mixture is at least 20% carbon or graphite by volume, the binder is at least 25% protein material by volume, and the solvent is saline solution.

3. The method of claim 1, wherein the protein material is selected from the group consisting of: albumin, gelatin and collagen.

4. The method of claim 1, wherein the drying includes baking at a temperature of at least 200° C.

5. The method of claim 4, wherein the drying includes:
drying at a temperature less than or equal to 200° C.; and
subsequently baking at a temperature greater than 200° C.

6. The method of claim 1, wherein the mixture is about 30% carbon powder or graphite powder by volume, the binder is about 35% protein material by volume, and the solvent is saline solution.

7. An electrode made by the method of claim 1.

8. An electrode for electrosurgery, comprising:
an electrically conductive substrate adapted for use as an electrosurgery electrode;
an electrically insulative layer on a portion of a surface of the electrode, but not on a tip or edge portion thereof; and
a coating on the tip or edge portion thereof, the coating being at least 10 μm thick and including carbon or graphite and a protein or denatured protein material,
wherein the coating supports a high voltage discharge of at least 1000 volts.

9. The electrode of claim 8, wherein the protein material is selected from the group consisting of: albumin, gelatin and collagen.

10. The electrode of claim 8, wherein the electrode is one of a ball, tube, screen, suction coagulator or forceps type.
